(19) United States
(12) Patent Application Publication
(10) Pub. No.: US 2016/0136424 A1
(43) Pub. Date: May 19, 2016

(54) TRANSCRANIAL PULSED CURRENT STIMULATION

(71) Applicant: BRAINIQUE AG, Stans (CH)

(72) Inventors: Raymond Watt, San Diego, CA (US); Jaen Frederik Labuschangne, Cape Town (ZA)

(73) Assignee: Brainique AG, Stans (CH)

(21) Appl. No.: 14/896,126

(22) PCT Filed: Jun. 9, 2014
(86) PCT No.: PCT/EP2014/061948

§ 371 Date: Dec. 4, 2015

Related U.S. Application Data
(60) Provisional application No. 61/832,743, filed on Jun. 7, 2013.

(51) Int. Cl. A61N 1/36

(52) U.S. Cl. CPC A61N 1/36025

(57) ABSTRACT

A computer-implemented method of providing a cranial electrotherapy stimulation program for use in a stimulation system is provided, the method comprising: generating a chaotic cranial electrotherapy stimulation program.

### TECHNICAL FIELD

This application relates to transcranial pulsed current stimulation.

### BACKGROUND

Noninvasive Electrical Brain Stimulation (herein referred to as NIEBS) applies gentle micro-current pulses to the brain using electrodes. It is widely accepted that NIEBS stimulates the brain to manufacture neurotransmitters. Noninvasive electrical brain stimulation has also been proposed for treatment of various medical conditions.

The signals operate to normalize the electrical output of the brain. NIEBS has thus been used/tested to treat substance dependence, depression and anxiety. It has been noted in at least some instances that NIEBS has equal or greater efficacy for the treatment of depression when compared to antidepressant medications, with fewer side effects.

The mechanism by which NIEBS produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

### SUMMARY

According to a first aspect, there is provided a computer-implemented method of providing a cranial electrotherapy stimulation program for use in a stimulation system, the method comprising: generating a chaotic cranial electrotherapy stimulation program.

Chaotic cranial electrotherapy stimulation, for example using transcranial pulsed current stimulation, has been shown to be particularly effective for the treatment of conditions such as depression when compared with treatments that are not chaotic.

According to a second aspect, there is provided a computer-implemented method of generating a transcranial pulsed current stimulation “TPCS” waveform, the method comprising generating a TPCS waveform based upon a chaotic cranial electrotherapy stimulation program provided by the first aspect.

By generating a TPCS waveform based upon a chaotic cranial electrotherapy stimulation program, an improved application of NIEBS can be effected and it is possible to increase the efficacy of noninvasive electrical brain stimulation (NIEBS).

According to a third aspect, there is provided a transcranial pulsed current stimulation [TPCS] generator comprising: a power source comprising an ac-to-dc converter for transforming alternating current signals into direct current; a current source with a controllable output; a digital-to-analog converter; a memory; and a microprocessor configured to execute instructions stored in memory, wherein the instructions further comprise: reading a prescription having parameters for a TPCS therapy from memory; and operating the digital to analog converter in conformance with the parameters read from memory, wherein the resultant analog signal is delivered to the current source.

By reading a prescription having parameters for a TPCS therapy and delivering an analog signal, an improved application of NIEBS can be effected and it is possible to provide a TPCS treatment that is configurable based upon parameters read from memory in a simple and effective manner.

According to a fourth aspect, there is provided a method for providing a transcranial pulsed current stimulation [TPCS] treatment regimen, comprising: providing a programmable NIEBS/TPCS generator configured to set operating parameters for TPCS treatment regimen based on an input parameter setting program; providing a menu of treatment options; providing a lookup table of parameters associated with at least one of the treatment options in said menu; selecting an option; selecting a set of TPCS parameters for a selected treatment option; and providing said set of TPCS parameters to the programmable NIEBS/TPCS generator.

By providing a menu of treatment options and allowing a user to select a treatment option, an improved application of NIEBS can be effected and it is possible to provide a breadth of options which are easily selectable by the operator. Furthermore, it is possible for the operator to produce a configurable TPCS treatment through a simple to use interface.

According to a fifth aspect, there is provided a method for providing a chaotic crainial electrotherapy stimulation program for use in a stimulation system, comprising: providing a programmable NIEBS generator configured for setting operating parameters for stimulation generation from a specified list of parameters [or from an input parameter setting program]; providing a menu for treatment options; providing a lookup table of available stimulation options; [all NIEBS options, tPCS, transcranial direct current stimulation tdcs, etc.] providing a random number generator; selecting a treatment option; operating the random number generator successively to select a group of parameters from the lookup table of stimulation options; and providing said selected group of parameters to a prescription generator, wherein said prescription generator supplies the selected parameters to the programmable NIEBS generator.

By providing a menu of treatment options and allowing a user to select a chaotic cranial electrotherapy stimulation program, an improved application of NIEBS can be effected and it is possible to provide a breadth of options for chaotic cranial electrotherapy treatment which is easily selectable by the operator. Furthermore, it is possible for the operator to produce a configurable treatment through a simple to use interface.

### BRIEF DESCRIPTION OF THE DRAWINGS

FIG. 1 illustrates block diagrams of example waveforms for use in a noninvasive electrical brain stimulation system in accordance with embodiments of the present technology.

FIG. 2 illustrates a block diagram of an example environment for a noninvasive electrical brain stimulation system in accordance with embodiments of the present technology.

FIGS. 3A, 3B, 3C, 3D, and 3E illustrate example tables for chaotic selection process in accordance with embodiments of the present technology.

FIG. 4 illustrates a flowchart for a chaotic selection process in accordance with embodiments of the present technology.

The drawings referred to in this description of embodiments should be understood as not being drawn to scale except if specifically noted.

### DETAILED DESCRIPTION

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

### Embodiments of Transcranial Pulsed Current Stimulation (TPCS)

The following describes 1) options for creating a transcranial pulsed current stimulation (TPCS) waveform; 2) a TPCS generator hardware description in two embodiments; 3) a method for selecting and delivering a TPCS treatment regimen; and 4) a method for selecting and delivering a Chaotic TPCS treatment regimen.

The following also describes the tables one might construct to make a wide range of TPCS service options available to a medical professional, and how to describe a suitable generator for implementing the selected parameters for the desired TPCS waveforms.

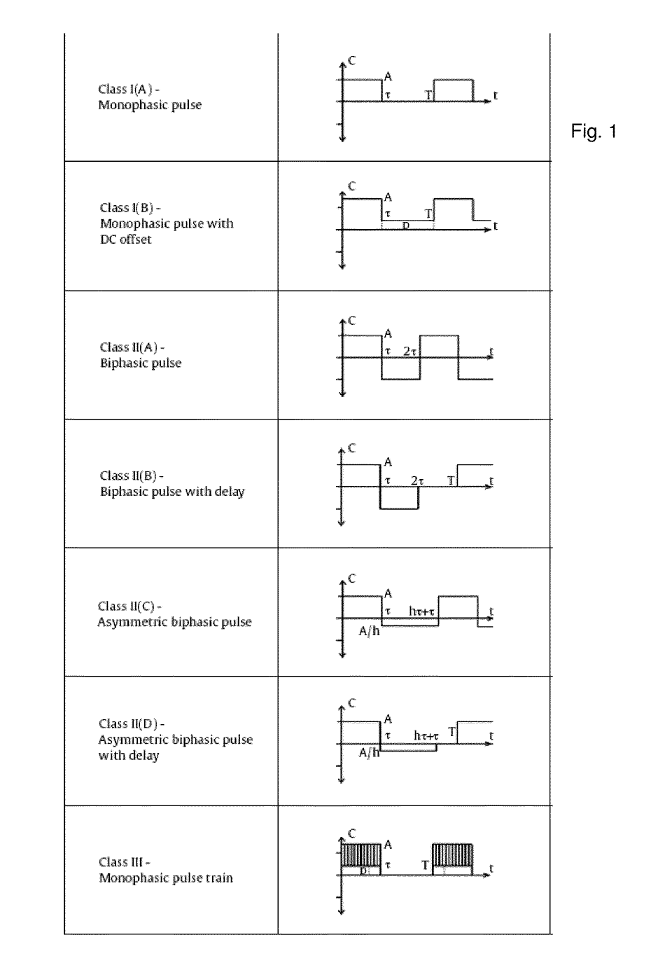

1. Transcranial pulsed current stimulation waveform options are shown in FIG. 1. They consist of the following options:

2. On-off pulses of positive voltage/current a finite duration and variable pulse duration and variable time spacing between a first pulse and a subsequent pulse. Thus there are two main timing variables:

pulse duration [on-time]

pulse separation [off-time, or delay time between stop of first pulse and start of next]

3. In addition, the amplitude of the pulse may be varied to suit a number of therapy treatments and the user's own preferences. As the voltage is increased, the amount of current delivered to the brain via the closed circuit between an active pair of skin contacts will increase. The amplitude of a preset, initial starting point for treatment may be arbitrarily set to a relatively low number, such as 0.1 or 0.55 volts, and may be adjustable up to 1.5 volts. This variable amplitude is often referred to as “intensity.”

The electrodes of the present technology may be attached to a user's body at any number of locations. For example, for TPCS, the electrodes are typically attached to the skin of the user's head and may be attached to the ears, earlobes, back of the skull, forehead, cheeks, etc. However, for both electrotherapy and TPCS in general the electrodes may attached anywhere on the body such as to fingers, the arms, legs, torso, head, etc.

3. A third variable may implemented as well. The pulse train may consist of alternating positive and negative pulses. The pulse pairs may be repeated with zero time delay between restart, or any other delay time period. The delay time period between restarts is also a significant variable in some treatment options. This method allows for reversing the direction of current passing thru the brain, according to a particular therapy protocol.

Pulse train configuration: positive only, or positive-negative.

4. The amplitude of the positive going pulse and the negative going pulse may not be equal. That is, the negative-going pulse may not need to have the same amplitude as the postive-going pulse.

So this is another variable: ratio of negative-going pulse amplitude to positive-going pulse amplitude. The advantage of this method is that the net direct current level passing through the brain may be varied from the maximum set by the average on-time of the positive-going pulse, to zero, when the amplitude of the positive and negative-going pulses and the pulse duration of each type of pulse, are equal. This allows for yet another variable in treatment therapy.

This ratio may be factory-set, or may be part of a therapy program adjustment.

5. A list of the variables and ranges

Single polarity pulse:

| | |
|---|---|
| pulse duration | 0.001 second to 10 seconds |
| pulse separation delay time | 0.001 second to unlimited. |
| pulse amplitude | 0.1 volt to 1.5 volts |

Alternating polarity pulse pair:

| | |
|---|---|
| pulse duration of positive pulse | 0.001 sec to 10 sec |
| pulse duration of negative-going pulse | 0.001 sec to 10 sec |
| pulse separation delay time | 0.001 sec to unlimited |
| pulse- negative-positive-amplitude ratio | 0 to 1.0 |
| pulse pair amplitude | 0.1 volt to 1.5 volts |

6. Treatment Time Period

The TPCS treatment period is yet another variable in the therapy program. For example, a treatment regimen of a series of pulses of 10 milliseconds may be delivered every 20 milliseconds for a period of 5 minutes, and then turned off for another 5 or 10 minutes. The treatment may be resumed again, with the same pulse duration and pulse separation time as above, or it may be changed.

### The TPCS Generator

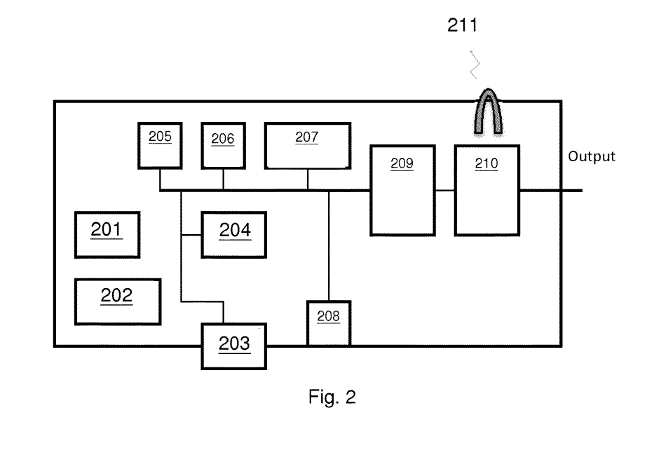

The TPCS generator is a self-powered device that implements either a fixed TPCS therapy program with preset parameters, or a programmable device that can receive a TPCS therapy program based on treatment options determined by a healthcare professional to be of use to a person with a specific condition. An embodiment of the TPCS generator is depicted in FIG. 2. The programmable device can receive a prescription for specific set of parameters selected from a table of options for the variables in the TPCS treatment method: pulse width/duration, delay time until the next pulse starts, pulse amplitude, or intensity, and pulse configuration, wherein the pulses may include positive-going and negative-going pulses. The generator comprises a microprocessor configured to 1) receive a prescription for a TPCS regimen from an external source, such as a memory stick or an internet connection to a server; 2) activate a set of instructions stored in memory to select parameters defined by the prescription to operate a digital-to-analog converter to create an appropriate TPCS waveform; convey this waveform to an output amplifier configured to operate as a current source; and provide an output level control for adjusting the voltage output from the current source, according to a user's preference.

Alternatively, the TPCS generator may be configured to simply read from memory a pre-programmed series of amplitudes defined according to a time sequence, and deliver the resultant waveform to the output amplifier 210. For example, a read-only memory may store a sequence of digital one's that when read out define a pulse having a duration of 20 milliseconds. The memory readout might comprise a series of digital 1's to be read out at 100 microsecond time steps, or 0.1 milliseconds. To form the 20 millisecond pulse, the microprocessor is clocked at 0.1 millisecond. If the pulse train were to be a continuous series of a plus 0.5 volt outputs alternating with a series of 0 volts, the next set of data stored in memory would be digital zeroes for the next 20 milliseconds. Such a read-only memory [ROM] system can be configured to create any number of combinations of pulse durations and pulse spacings, but only one may operate per ROM. Multiple ROM's can be included in the generator. It is less convenient to use the ROM system if there is a therapy regimen that requires having a variable pulse component, such as the delay time between pulses. In such cases, the programmable generator has more utility.

The TPCS generator depicted by FIG. 2 shows a battery 201 to supply power, an AC/DC converter 202, RAM 205 and ROM 206, a memory card that may be an SD card 203 or other removable memory or may be memory that is hardwired into the TPCS generator. The device also comprises a processor 207 and a data port 208 as well as a digital to analog (D-A) converter 209. The amps 210 associated with the TPCS may be controlled by controlling the intensity or amplitude of the pulses. The intensity control 211 in FIG. 2 represents a control wheel which may be adjusted by a user. Other variables may also be controlled by a user. Such controls may be actual physical buttons or wheels or may be controlled via a software interface or controlled via another device in communication with the generator using the data port 208. The TPCS generator may also include a ROM2 204 and an output,

### The Chaotic TPCS System

The chaotic system is named for varying many of the pulse characteristics shown above in “5. A list of the variables and ranges” in a random, non-repetitive process. For example, the pulse duration may be varied over a range of 2 seconds to 0.002 seconds. Similarly, the time period between a first single pulse and the next single pulse may vary from 0.5 second to as high as a pulse every millisecond. The pulse train may shift from only positive-going pulses to both positive and negative. The relative amplitude of positive to negative pulses may be varied.

The Chaotic TPCS System may be implemented using a random-number generator to select a parameter from among the parameters listed above in “5. A list of the variables and ranges”. The random number generator can be conditioned to choose from a limited set of options, where all are equally probable.

For example, an initial series of positive-going pulses of a selected amplitude [intensity] may be delivered using a pulse duration of 10 milliseconds and a repetition rate of 50 pulses per second. This corresponds to an equal on/off time between pulses, and a Direct Current average of 50% of maximum for a given intensity [applied voltage level.] Such a treatment regimen may be applied for 5 minutes continuously, and then turned off for another time period, such as 10 minutes. In this case, the pulse duration is 10 milliseconds, and the pulse delay between pulses is set to 10 milliseconds, and the treatment period is set to 5 minutes. But after some initial time delay, as selected by a random process, a new treatment formulation may be introduced. For example, the pulse duration may be extended to 100 milliseconds, with a pulse delay time of 1 second, this time delivered for a 2 minute period.

Thus a Chaotic Treatment Regimen may consist of a programmable set of parameters as shown above in “5. A list of the variables and ranges,” along with a variable called Treatment Duration. The choice for each variable may be selected from the output of a random number generator, normalized to the particular range found suitable for the treatment recommended for the user's condition. The random number generator adjusts each parameter according to a treatment duration selection, also selected by a random number generator.

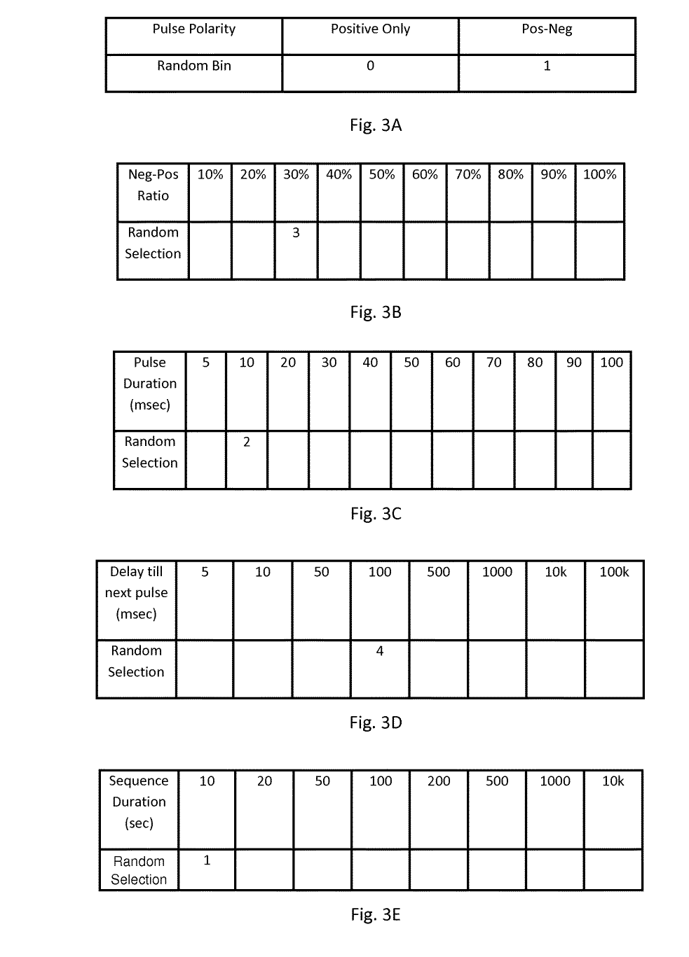

With reference to FIG. 3A to 3E which illustrate an example of a chaotic selection process. The tables shown in FIG. 3A to 3E may be described as rolling a dice where the numbers add up to ten. In such an example you could also include more than two dice. However, in the present technology, instead of rolling dice, a random number generator operates in place of rolling dice. Additionally, there may be constraints on the allowable combinations of results. For example, experimental data may demonstrate that 100% is not a viable option for the Neg-Pos ratio. Therefore, the allowable combinations of results are constrained so that 100% is not an allowed option for any combination. In the example of FIGS. 3A to 3E, the pulses are positive-negative with a negative ratio of 30% amplitude of positive, pulse duration is 10 msec, delay until next pulse is 100 msec, and sequence lasts for 10 seconds.

Random Number Generator for use with the Chaotic Treatment Resource

A random number generator may be realized in software that may run on the server which is used to support the construction of suitable TPCS therapy configurations. Many are freely available from resources on the internet. Suppliers include Intelligent Masters at http://geocities.com/intelligentmasters.Utilities, en.softonic.com with their random-number-generator.en.softonic.com, or Random Number Generator Pro from en.kioskea.net. The Java programming language has a resource for generating random numbers in the Java Utility package.

Pseudo-random number generators can also be used. Random number generators may also be realized in physical hardware designed for this purpose. See Wikipedia for a list of pseudo-random number generator algorithms, and for hardware-based True random number generators. Generator services are also available online from a variety of web sites such as HotBits, random.org, EntropyPool, or randomnumbers.info.

Random Number Generator

A random number generator may be realized in software that may run on the server which is used to support the construction of suitable TPCS therapy configurations. Many are freely available from resources on the internet. Suppliers include Intelligent Masters at http://geocities.com/intelligentmasters.Utilities, en.softonic.com with their random-number-generator.en.softonic.com, or Random Number Generator Pro from en.kioskea.net. The Java programming language has a resource for generating random numbers in the Java Utility package.

Pseudo-random number generators can also be used. Random number generators may also be realized in physical hardware designed for this purpose. See Wikipedia for a list of pseudo-random number generator algorithms, and for hardware-based True random number generators. Generator services are also available online from a variety of web sites such as HotBits, random.org, EntropyPool, or randomnumbers.info.

Embodiments of the present technology are for systems and methods for Noninvasive electrical brain stimulation. Noninvasive electrical brain stimulation (NIEBS) is a treatment that applies pulses to the brain across the head of the patient using electrodes. There are many types of NIEBS such as transcranial direct current stimulation (tDCS) which is a form of neuro-stimulation which uses constant, low current delivered directly to the brain area of interest via small electrodes. There are three different types of tDCS: anodal, cathodal, and sham. The anodal stimulation is positive (V+) stimulation that increases the neuronal excitability of the area being stimulated. Cathodal (V−) stimulation decreases the neuronal excitability of the area being stimulated. Cathodal stimulation can treat psychological disorders that are caused by the hyper-activity of an area of the brain. Sham stimulation is used as a control in experiments. Sham stimulation emits a brief current but then remains off for the remainder of the stimulation time. With sham stimulation, the person receiving the tDCS does not know that they are not receiving prolonged stimulation.

Another form of NIEBS is transcranial alternating current stimulation (tACS) which is a noninvasive means by which alternating currents applied through the skull over the occipital cortex of the brain entrains in a frequency-specific fashion the neural oscillations of the underlying brain. Another class of NIEBS is transcranial pulsed current stimulation (tPCS).

Transcranial magnetic stimulation (TMS) is a noninvasive method to cause depolarization or hyperpolarization in the neurons of the brain. TMS uses electromagnetic induction to induce weak electric currents using a rapidly changing magnetic field; this can cause activity in specific or general parts of the brain with minimal discomfort, allowing the functioning and interconnections of the brain to be studied. A variant of TMS is repetitive transcranial magnetic stimulation (rTMS).

The present technology is not limited to one form of NIEBS. Therefore, as used herein, NIEBS may refer to many varieties of NIEBS including tDCS, tACS, tPCS, TMS, rTMS, and any other neuro-stimulation type protocols.

NIEBS involves brain stimulation by low current low voltage that may use alternating square waves or other waves. The effect is to improve the brain's “plasticity,” making it easier to learn. The effect may also be described as an increase in focus, getting into the flow, or being in the zone.

The present technology employs hardware for NIEBS that attaches electrodes to the head of the patient. The hardware may also include speakers such as headphones. The present technology may apply NIEBS to a user and may or may not simultaneously play audio for the user via speakers such as headphones. The pulse for the NIEBS may or may not be based on the rhythm or beat of the audio signal. The speakers may or may not be combined into one frame or housing with the electrodes.

The NIEBS treatment may be adjusted, modified or controlled by a user or patient. For example, a user may control the intensity of amplitude of the treatment. The user may develop levels of control that are preferred by the user. Such levels of control may be described as a NIEBS Control Profile. The user may wish to share her NIEBS Control Profile with other users or share other information regarding a NIEBS Control Profile. Other information may be reviews, feedback, or blogs regarding NIEBS Control Profiles. For example, a user may post a NIEBS Control Profile with a review of the profile. A second user may then download the NIEBS Control Profile and offer feedback or comments. Such forums may be public or private.

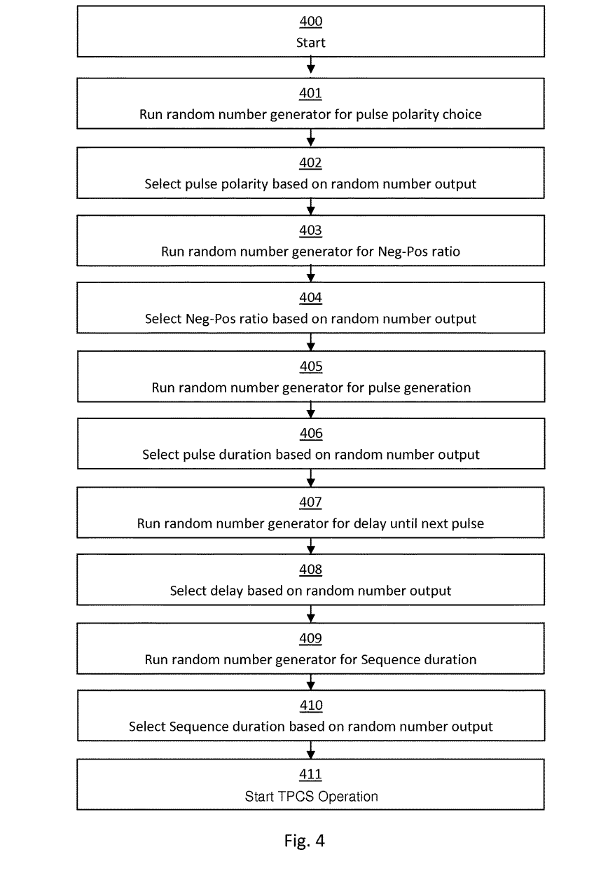

With reference to FIG. 4 which depicts a flowchart in accordance with embodiments of the present technology which is a flowchart for a chaotic selection process. Steps 400-411 illustrate an example flowchart based on the above described techniques. FIG. 4 may be used for TPCS and may be a computer implemented method that is carried out by processors and electrical components under the control of computer usable and computer executable instructions.

With reference to FIG. 1 which depicts wave forms that may be employed for use with the present technology. A NIEBS generator may receive wave forms from an audio source or from a waveform synthesizer associated with the NIEBS generator. The NIEBS generator may generate a NIEBS signal with associated wave forms for the NIEBS treatment. FIG. 1 depicts well known square wave forms for use in the present technology. The present technology is not limited to wave forms in FIG. 1 but may also employ other wave forms such as sine waves.

Wave forms for the present technology may be stored in a library and are used to create pulse patterns or pulse trains for use in NIEBS. The wave forms may be implemented via a programmable D/A converter. Research indicates that different pulse patterns have different effects on the brain, and that some pulse patterns have different effects on various conditions. Therefore, there is a need for a library of different pulse patterns to suit different health conditions.

The rate of pulses per second refers to a start of positive-going pulse to stop, with the delay until the next positive-going pulse starts. Like a sine wave, regardless of whether or not there is a negative-going pulse. “Beginning of a pulse rising, to the next time the pulse starts rising again.” The following are examples of pulse rates that may be employed by the present technology:

1. Pulse rate in range of 3-5 Hz. Low Freq.

2. Pulse rate in range of 50-100 Hz. Low Freq.

3. Pulse rate in range from 100-640 Hz. High Freq.

4. Pulse rate in range of 0.1-100 Hz

5. Direct current

Current level delivered: 1.5 mA. [milli-Ampere]

Current density on the skin: safety limit is between 25 and 60 microA/cm² [from Poreisz et al., 2007] The electric field across the brain tissue is on the order of less than 5 mV/mm, or 5 milli-Volts/millimeter.

Pulse pattern may be a Random Noise Stimulation pattern. Good results reported by Fertonani et al in paper “Random Noise Stimulation Improves Neuroplasticity in Perceptual Learning,” The Journal of Neuroscience, Oct. 26, 2011 31(43):15416-15423.

Noninvasive electrical brain stimulation (herein referred to as NIEBS) applies gentle micro-current pulses to the brain using electrodes. The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

In NIEBS significant amounts of current pass the skull and reach cortical and subcortical structures. In addition, depending on the montage, induced currents at subcortical areas, such as midbrain, pons, thalamus and hypothalamus are of similar magnitude than that of cortical areas. Incremental variations of electrode position on the head surface also influence which cortical regions are modulated. The high-resolution modeling predictions suggest that details of electrode montage influence current flow through superficial and deep structures. Also, laptop based methods for tPCS dose design using dominant frequency and spherical models. These modeling predictions and tools are the first step to advance rational and optimized use of tPCS and NIEBS.

It is widely accepted that NIEBS stimulates the brain to manufacture neurotransmitters, like endorphins, which improve moods, emotions and cognitive capabilities. Noninvasive electrical brain stimulation has also been proposed for treatment following a stroke, brain trauma, high blood pressure, and Alzheimer's disease, as well as any or all neurological disorders, any or all mental disorders, and any or all cognitive enhancements. The present technology may also be used by healthy users or users who are not suffering from any diagnosed disorders or diseases. For example, a healthy user may be a student using the present technology to increase focus and learning abilities or may be an athlete using the present technology to increase sports performance.

The signals apparently normalize the electrical output of the brain. NIEBS has thus been used or tested to treat substance dependence, depression and anxiety. It has been noted in at least some instances that NIEBS has equal or greater efficacy for the treatment of depression when compared to antidepressant medications, with fewer side effects. NIEBS may be used specifically in combination with anti-depressant drugs and may be used to eliminate the side effects of central nervous system (CNS) medications or drugs in general. NIEBS may also be used in conjunction with other traditional medicine.

Treatments can be used in association with the present technology in ranges from less than one second up to an infinite number of seconds. The present technology is not limited to a particular range of duration, current, or frequency. The following ranges are meant as examples and do not limit the present technology. In one embodiment, a range is used from 10 to 30 minutes in duration although the treatments may extend up to 11/2 hours depending on the electrical current configuration. The currents employed may be applied in pulse form or direct form with a pulse width in the range of from about 1 to about 500 milliseconds (ms) at a frequency of from about 0.1 Hertz (Hz) up to 1000 Hz with the current being less than 1 milliampere (mA) up to 5 mA

In accordance with an embodiment of the invention there is provided equipment for the implementation of a method as defined above, said equipment comprising a noninvasive electrical brain stimulation pulse generator and associated electrodes for applying pulses generated by the pulse generator to the head of a patient, wherein the equipment includes multiple electrodes.

In an embodiment of the invention, there is an audio signal player and at least one associated loudspeaker for converting output from the signal player into audible sound. The at least one loudspeaker is preferably a pair of earphones and the noninvasive electrical brain stimulation pulse generator and sound signal generator may be built into a single unit, but are not necessarily thus combined.

Note that there are the following types of stimulation configurations:

1. Positive going pulse, with a direct current average in one direction. Class 1A and Class 1B deliver a varying amount of direct current in little bursts.

2. Alternating current pulses, where the direction of current alternates from positive going to negative going, as in Class IIA and Class IIB and IIC and IID. The average may be in one direction predominantly, or may average out to zero if the pulses are symmetric and equal in duration over time. You can see that for some modes, there is a net direct current passing thru the brain.

3. Class III shows a pulse train with a delay between delivery of a series of pulses.

The next paragraphs discuss how this delay may be configured, and is part of the overall therapy formulation that is available to a medical practitioner.

1. Random time period. Use a random number generator with a specified range in seconds. For example, 1-100 seconds. Run the random number generator which is set to produce a number between 1 and 100. Use that number as the time period between pulses. Run the generator after each pulse to determine the next time delay, or period, from the last pulse.

2. Semi-random time period.

Pick some time periods that are known to have some therapeutic effect. Make a table. For example:

Random No. 1 3 5 10 20 40 60 100.

Bin containing 1 2 3 4 5 6 7 8

the delay

Then randomly select from this group of time periods. Again, use a random number generator whose bounds are the number of allowed states. In the above example, there are 8 possible delay time periods. Set the random number generator to select any of the numbers from 1 to 8. Use the time delay associated with that bin number.

Say the random number generator picks 4. That means we use 10 second delay as the time period to the next pulse train initiation.

3. Periodic but increasing delay, with a plan

Here the time delay from one pulse train event to the next is arbitrarily set to predetermined sequence. It may be one with a set increase from one period duration to the next. As in 5 10 30 60 repeat 5 10 30 60.

4. Periodic, static period

Set delay to one of the group [1, 2, 3, 4, 5, 6, 7, 8, 9, 10] seconds. Or any other time period from 1 to 300 seconds, for example.

5. Continuous pulse train with no delay between any arbitrary group of pulses. Arbitrary duration of such pulse trains, selected from group [1-1000] seconds.

6. Direct Current Stimulation

No pulses, just application of a constant voltage for some time period. One could consider this a special case of a single positive going pulse with a really long time duration.

Notes on using chaotic/random pulse for NIEBS:

Pulses or pulse trains for NIEBS and NIEBS prescriptions may be patterned or random. However, the idea of random pulses may not be desirable as random may still indicate a measureable structure impulse. The term chaotic pattern is better description of the pulse referred to herein. Chaotic may also be used to define the variety of the pauses or periods in between pulse trains.

### Computer Implemented Methods

It should be appreciated that the methods described herein may be computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of computer usable storage medium. In one embodiment, the methods may reside in a computer usable storage medium having instructions embodied therein that when executed cause a computer system to perform the method. In one embodiment, the NIEBS signals described herein are non-transitory but rather are sent over wired connections to the electrodes.

It is intended that the foregoing detailed description be regarded as illustrative rather than limiting and that it be understood that the detailed description should not be used to limit the scope of the invention.

1. A computer-implemented method of providing a cranial electrotherapy stimulation program for use in a stimulation system, the method comprising:
generating a chaotic cranial electrotherapy stimulation program.

2. The method of claim 1, wherein generating the chaotic cranial electrotherapy stimulation program comprises selecting pulse characteristics in a non-repetitive manner.

3. The method of claim 2, wherein the pulse characteristics are selected in a random manner.

4. The method of claim 3, wherein the pulse characteristics are selected by operating a random number generator successively to select a group of parameters from a lookup table of stimulation options.

5. The method of claim 4, further comprising
providing a menu for treatment options;
providing the lookup table of available stimulation options;
selecting a treatment option;
providing said selected group of parameters to a prescription generator, wherein said prescription generator supplies the selected parameters to a programmable NIEBS generator.

6. The method of claim 2, wherein the pulse characteristics include at least one of: pulse duration, pulse separation delay time, pulse amplitude, pulse duration of positive pulse, pulse duration of negative-going pulse, pulse negative-positive-amplitude ratio, and pulse pair amplitude.

7. The method of claim 4 wherein the set of parameters comprise at least one of:
a single polarity pulse having a pulse duration, wherein the pulse duration is selected from the group comprising 0.001 second to 10 seconds, 0.001 intervals;
a pulse separation delay time wherein the separation is selected from the group comprising 0.001 second to 10,000 seconds; and
a pulse amplitude wherein the range of amplitude is selected from the range comprising 0.1 volt to 1.5 volts.

8. A computer-implemented method of generating a transcranial pulsed current stimulation “TPCS” waveform, the method comprising generating a TPCS waveform based upon a chaotic cranial electrotherapy stimulation program provided by the method of any preceding claim.

9. The method of claim 8, wherein the TPCS waveform is generated by a programmable TPCS generator.

10. The method of claim 9 wherein the programmable TPCS generator comprises an AC-to-DC converter for powering the generator from an audio source.

11. The method of claim 9 wherein the pulse amplitude may be manually adjusted via an external control on the TPCS generator.

12. The method of claim 8 further comprising applying the generated TPCS waveform to a patient.

13. A computer-readable medium comprising computer-readable instructions to implement the method of claim 1.

14. An apparatus configured to perform the method of claim 1.

15. A transcranial pulsed current stimulation [TPCS] generator comprising:
a power source comprising an ac-to-dc converter for transforming alternating current signals into direct current;
a current source with a controllable output;
a digital-to-analog converter;
a memory; and
a microprocessor configured to execute instructions stored in memory, wherein the instructions further comprise:
reading a prescription having parameters for a TPCS therapy from memory; and
operating the digital to analog converter in conformance with the parameters read from memory, wherein the resultant analog signal is delivered to the current source.

16. A method for providing a transcranial pulsed current stimulation [TPCS] treatment regimen, comprising:
providing a programmable NIEBS/TPCS generator configured to set operating parameters for TPCS treatment regimen based on an input parameter setting program;
providing a menu of treatment options;
providing a lookup table of parameters associated with at least one of the treatment options in said menu;
selecting an option;
selecting a set of TPCS parameters for a selected treatment option; and
providing said set of TPCS parameters to the programmable NIEBS/TPCS generator.

17. The method of claim 13 wherein the step of providing the set of TPCS parameters to the programmable NIEBS/TPCS generator further comprises:
providing said selected group of parameters to a prescription generator, wherein said prescription generator supplies the selected parameters to the programmable NIEBS generator.

18. The method of claim 13 wherein the programmable NIEBS/TPCS generator comprises an ac-to-dc converter for powering the generator from an audio source.

19. The method of claim 13 wherein the set of parameters comprises:
a single polarity pulse having a pulse duration wherein the duration is selected from the group comprising 0.001 second to 10 seconds, 0.001 intervals;
a pulse separation delay time wherein the separation is selected from the group comprising 0.001 second to 10,000 seconds; and
a pulse amplitude wherein the range of amplitude is selected from the range comprising 0.1 volt to 1.5 volts.

20. The method of claim 16 wherein the pulse amplitude may be manually adjusted via an external control on the TPCS generator.

21. A method for providing a chaotic cranial electrotherapy stimulation program for use in a stimulation system, comprising:
providing a programmable NIEBS generator configured for setting operating parameters for stimulation generation from a specified list of parameters for from an input parameter setting program;
providing a menu for treatment options;
providing a lookup table of available stimulation options;
providing a random number generator;
selecting a treatment option;
operating the random number generator successively to select a group of parameters from the lookup table of stimulation options; and
providing said selected group of parameters to a prescription generator, wherein said prescription generator supplies the selected parameters to the programmable NIEBS generator.
